SEQUENCE LISTING (24 sequences)

SEQ ID NO: 1 - DNA, 20 bp, Artificial
  RT-PCR primer
acggcgagaacatcttctgg

SEQ ID NO: 2 - DNA, 20 bp, Artificial
  RT-PCR primer
cgccgaggttgttgtcgcag

SEQ ID NO: 3 - DNA, 19 bp, Artificial
  RT-PCR primer
aggtgaacggcagcgatcc

SEQ ID NO: 4 - DNA, 21 bp, Artificial
  RT-PCR primer
acccgtcttggcaaggtacag

SEQ ID NO: 5 - DNA, 17 bp, Artificial
  RT-PCR primer
accccgactcttccctt

SEQ ID NO: 6 - DNA, 21 bp, Artificial
  RT-PCR primer
atgatgttggtgatcacgccg

SEQ ID NO: 7 - DNA, 17 bp, Artificial
  RT-PCR primer
cccgcggccaggaatcg

SEQ ID NO: 8 - DNA, 18 bp, Artificial
  RT-PCR primer
ggccctgctgcactccca

SEQ ID NO: 9 - DNA, 20 bp, Artificial
  RT-PCR primer
gccagtgggcggtggtaggc

SEQ ID NO: 10 - DNA, 20 bp, Artificial
  RT-PCR primer
atgccgatgttgaacccgtc

SEQ ID NO: 11 - DNA, 21 bp, Artificial
  RT-PCR primer
gactggcacaatctggcaccc

SEQ ID NO: 12 - DNA, 19 bp, Artificial
  RT-PCR primer
cttggccacactcccaccg

SEQ ID NO: 13 - DNA, 23 bp, Artificial
  RT-PCR primer
gctgttttccctagcattgttgg

SEQ ID NO: 14 - DNA, 23 bp, Artificial
  RT-PCR primer
ataagagaatccgtgagatcccg

SEQ ID NO: 15 - DNA, 21 bp, Artificial
  RT-PCR primer
gcttggagtcctgcttggacg

SEQ ID NO: 16 - DNA, 23 bp, Artificial
  RT-PCR primer
cgcagttaagaggactgtccggc

SEQ ID NO: 17 - DNA, 20 bp, Artificial
  RT-PCR primer
aagggttcacaaccaggcac

SEQ ID NO: 18 - DNA, 19 bp, Artificial
  RT-PCR primer
cactgcatgggacctacgc

SEQ ID NO: 19 - DNA, 21 bp, Artificial
  RT-PCR primer
gggacggctctcgtggctacc

SEQ ID NO: 20 - DNA, 22 bp, Artificial
  RT-PCR primer
cgcgcgttatcgaaactcgcgg

SEQ ID NO: 21 - DNA, 21 bp, Artificial
  RT-PCR primer
gacgccgaccgtgccgccggg

SEQ ID NO: 22 - DNA, 22 bp, Artificial
  RT-PCR primer
cggcgactctcccgtcttggcc

SEQ ID NO: 23 - DNA, 22 bp, Artificial
  RT-PCR primer
ggaccaatgcagcaacggaggc

SEQ ID NO: 24 - DNA, 23 bp, Artificial
  RT-PCR primer
ggctgcccaatgagctcattgcc

(19) United States
(12) Patent Application Publication
(10) Pub. No.: US 2013/0091606 A1
(43) Pub. Date: Apr. 11, 2013

(54) Methods and compositions for enhanced Agrobacterium-mediated transformation Efficiency

(71) Applicants: Wanjun Zhang, Beijing (CN); Rongda Qu, Holly Springs, NC (US); Ralph E. Dewey, Apex, NC (US)

(72) Inventors: Wanjun Zhang, Beijing (CN); Rongda Qu, Holly Springs, NC (US); Ralph E. Dewey, Apex, NC (US)

(73) Assignee: BAYER CROPSCIENCE LP, Research Triangle Park, NC (US)

(21) Appl. No.: 13/531,424

(22) Filed: Jun. 22, 2012

Related U.S. Application Data
(60) Provisional application No. 61/500,681, filed on Jun. 24, 2011.
(60) Provisional application No. 61/583,355, filed on Jan. 5, 2012.

(51) Int. Cl. C12N 15/82

(52) U.S. Cl. CPC C12N 15/8205
USPC 800/294; 435/431

(57) ABSTRACT

The present invention relates to a method of Agrobaterium-based transformation of a plant comprising: generating a callus explant by, performing callus induction and maintenance in a plant in at least a first solution comprising less than about 100 mg/l of inositol; subjecting the callus explant to a cold treatment in a second solution comprising glutamine; and co-cultivating the callus explant with an Agrobacterium in a third solution comprising glutamine. The present invention also relates to a callus induction medium and/or callus maintenance medium, comprising: less than about 100 mg/l of inositol, and optionally at least one constituent selected from the group consisting of: an N6 salt or any plant culture media, proline, casein hydrolysate, a B-Vitamin, maltose, phytagel, 2,4-D, BAP and/or other plant growth regulators/hormones.

### CROSS REFERENCE TO RELATED APPLICATION

This application claims priority to provisional application No. 61/500,681, filed on Jun. 24, 2011, and No. 61/583,355, filed on Jan. 5, 2012, and the contents of each of these applications are incorporated herein by reference in their entireties.

### BACKGROUND

1. Field

The present invention relates to methods of introducing genes into plants, specifically Agrobacterium-mediated transformation methods.

2. Description of Related Art

In molecular biology transformation is the genetic alteration of a cell resulting from the direct uptake, incorporation and expression of exogenous genetic material (exogenous DNA) from its surrounding and taken up through the cell membrane(s). Transformation occurs most commonly in bacteria and in some species occurs naturally. Transformation can also be effected by artificial means. Bacteria that are capable of being transformed, whether naturally or artificially, are called competent. Transformation is one of three processes by which exogenous genetic material may be introduced into a bacterial cell, the other two being conjugation (transfer of genetic material between two bacterial cells in direct contact), and transduction (injection of foreign DNA by a bacteriophage virus into the host bacterium). Transformation may also be used to describe the insertion of new genetic material into nonbacterial cells including animal and plant cells.

Agrobacterium mediated transformation is the easiest and most simple form of plant transformation. Plant tissue (often leaves) are cut into small pieces, e.g. 10×10 mm, and soaked for 10 minutes in a fluid containing suspended Agrobacterium. Some cells along the cut will be transformed by the bacterium that inserts its DNA into the cell. Placed on selectable rooting and shooting media, the plants will regrow. It is well known that Agrobacterium is a genus of Gram-negative bacteria that uses horizontal gene transfer to cause tumors in plants. Agrobacterium tumefaciens is the most commonly studied species in this genus. Agrobacterium is well known for its ability to transfer DNA between itself and plants, and for this reason it has become an important tool for genetic engineering.

A. tumefaciens causes crown-gall disease in plants, which is characterized by a tumour-like growth or gall on the infected plant, often at the junction between the root and the shoot. Tumors are incited by the conjugative transfer of a DNA segment (T-DNA) from the bacterial tumour-inducing (Ti) plasmid. The closely related species, A. rhizogenes, induces root tumors, and carries the distinct Ri (root-inducing) plasmid. The taxonomy of Agrobacterium can be generalized by the 3 biovars that exist within the genus, A. tumefaciens, A. rhizogenes, and A. vitis. Strains within A. tumefaciens and A. rhizogenes are known to be able to harbour either a Ti or Ri-plasmid, whilst strains of A. vitis, generally restricted to grapevines, can harbor a Ti-plasmid. Non-Agrobacterium strains have been isolated from environmental samples that harbor a Ri-plasmid whilst laboratory studies have shown that non-Agrobacterium strains can also harbor a Ti-plasmid. Many environmental strains of Agrobacterium do not possess either a Ti or Ri-plasmid. These strains are avirulent.

The plasmid T-DNA is integrated semi-randomly into the genome of the host cell, and the tumor morphology genes on the T-DNA are expressed, causing the formation of a gall. The T-DNA carries genes for the biosynthetic enzymes for the production of unusual amino acids, typically octopine or nopaline. It also carries genes for the biosynthesis of the plant hormones, auxin and cytokinins. These plant hormones produce opines, providing a carbon and nitrogen source for the bacteria that most other microrganisms cannot use, giving Agrobacterium a selective advantage. By altering the hormone balance in the plant cell, the plant cannot control the division of those cells, and tumors form. The ratio of auxin to cytokinin produced by the tumor genes determines the morphology of the tumor (root-like, disorganized or shoot-like).

The ability of Agrobacterium to transfer genes to plants and fungi is used in biotechnology, in particular, genetic engineering for plant improvement. A modified Ti or Ri plasmid can be used. The plasmid is ‘disarmed’ by deletion of the tumor inducing genes; the only essential parts of the T-DNA are its two small (25 base pair) border repeats, at least one of which is needed for plant transformation. Advancements in the field have let to the development of methods to alter Agrobacterium into an efficient delivery system for gene engineering in plants.

The genes to be introduced into the plant are cloned into a plant transformation vector that contains the T-DNA region of the disarmed plasmid, together with a selectable marker (such as antibiotic resistance) to enable selection for plants that have been successfully transformed. Plants are grown on media containing a selection agent, such as an antibiotic or an herbicide following transformation, and those that do not have the T-DNA integrated into their genome will die. An alternative method is agroinfiltration. Transformation with Agrobacterium can be achieved in two ways. Protoplasts, or leaf-discs can be incubated with the Agrobacterium and whole plants regenerated using plant tissue culture. A common transformation protocol for Arabidopsis is the floral-dip method: the flowers are dipped in an Agrobacterium culture, and the bacterium transforms the germline cells that make the female gametes. The seeds can then be screened for antibiotic resistance (or another marker of interest), and plants that have not integrated the plasmid DNA will die.

Although several different methodologies have been developed to introduce foreign genes into plants [e.g. biolistics-based transformation, electroporation of protoplasts, silicon fibers], Agrobacterium-mediated transformation continues to be the technique of choice for most plant species. Agrobacterium-based protocols typically offer the following advantages with respect to alternative technologies: [1] simplicity of use; [2] no need for expensive, specialized equipment; [3] higher transformation efficiencies; [4] relatively low transgene copy number; and [5] higher percentage of intact copies of the construct (since the ends are usually defined by the right and left borders of the T-DNA). Due to the widespread popularity of Agrobacterium-mediated transformation, there is a large body of literature describing a myriad of protocol permutations designed to enhance the efficiencies of transgene integreation across a wide range of plant species.

Several groups have previously reported success in generating transgenic perennial ryegrass plants using Agrobacterium (Wu et al. Salt-tolerant transgenic perennial ryegrass (Lolium perenne L.) obtained by Agrobacterium tumefaciens-mediated transformation of the vacuolar Na+/H+ antiporter gene. 2005 Plant Sci. 169: 65-73.; Altpeter, F. Perennial ryegrass L. perenne L. 2006. In Methods in Molecular Biology, Vol. 344: Agrobacterium Protocols. K. Wang, ed., Humana Press Inc., Totowa, N.J. pp. 55-64; Bajaj et al. A high throughput Agrobacterium tumefaciens-mediated transformation method for functional genomics of perennial ryegrass (Lolium perenne L.) 2006. Plant Cell Rep. 25: 651-659; Cao et al. Transformation of recalcitrant turfgrass cultivars through improvement of tissue culture and selection regime. Plant Cell Tiss. Organ Cult. 85: 307-316, 2006; Sato and Takamizo, Agrobacterium tumefaciens-mediated transformation of forage-type perennial ryegrass (Lolium perenne L.). 2006 Grassland Sci. 52: 95-98; Wu et al. Efficient regeneration and Agrobacterium-mediated stable transformation of perennial ryegrass. 2007. Russ. J. Plant Physiol. 54: 524-529, 2007). Cao et al. (2006) reported perennial ryegrass transformation efficiencies as high as 23% (defined as [#transgenic plants recovered/#calluses exposed to agro-infection]×100%) after testing the effects of a variety of light, temperature, and antioxidant conditions at different steps. Wu et al. (2005) reported a 20% transformation efficiency for perennial ryegrass after optimizing the timing of acetosyringone exposure and including an osmotic treatment. Other publications describing perennial ryegrass transformation reported efficiencies of 16% or less (Altpeter, 2006; Bajaj et al. 2006; Sato and Takamizo, 2006; Wu et al. 2007).

There remains a need to improve transformation efficiencies in commercially useful plants including but not limited to perennial ryegrass and rice.

### BRIEF SUMMARY

The present invention comprises a method of Agrobacterium-based transformation that dramatically improves the transformation efficiency.

The present invention comprises a method for Agrobacterium-based transformation of plants that increases the rate of transgene incorporation comprising modifications in the inositol and glutamine content of growth media in combination with a cold treatment.

The present invention comprises a set of conditions that greatly enhance transformation efficiencies. The specific alterations that resulted in the increased rate of transgene incorporation, modifications in the inositol and glutamine content of the growth media coupled with a cold treatment, have not been reported previously. The results of experimental data suggest the application of these conditions will make it possible to obtain transgenic perennial ryegrass plants at a much higher efficiency than that attained by others. Furthermore, by applying the same set of transformation conditions with the transformation of rice suspension cultures, similar enhancement in transformation efficiency was observed, suggesting the application of this set of conditions to the broad application in the field of plant transformation.

An embodiment of the present invention is directed to a method of Agrobaterium-based transformation of a plant comprising: generating a callus explant by performing callus induction and maintenance in a plant in at least a first solution comprising less than about 100 mg/l of inositol; subjecting the callus explant to a cold treatment in a second solution comprising glutamine; and co-cultivating the callus explant with an Agrobacterium in a third solution comprising glutamine.

A second embodiment of the present invention is direct to a callus induction medium and/or callus maintenance medium, comprising: less than about 100 mg/l of inositol, and optionally at least one constituent selected from the group consisting of: an N6 salt or any plant culture media, proline, casein hydrolysate, a B-Vitamin, maltose, phytagel, 2,4-D, BAP and/or other plant growth regulators/hormones.

### BRIEF DESCRIPTION OF THE DRAWINGS

FIG. 1 shows enhanced perennial ryegrass transformation by combined treatments. FIG. 1(a) shows that cold-shock treatment prevented calluses from browning after Agrobacterium infection. The calluses were cultured on medium containing myo-inositol (MI) used in the experiments. Photos were taken after 4 days of co-cultivation. FIG. 1(b) shows factors that affect perennial ryegrass transformation. Four-month-old calluses are cultured on medium supplied with or without 100 mg/l myo-inositol (w/ I or w/o I). The calluses were either treated with a cold shock (Cold+) or not (Cold−) for 20 min in 3% maltose solution on ice prior to infection. Addition of 100 μM L-Gln to the 3% maltose solution and bacterium suspension medium (Gln+) or not (Gln−) is another factor tested. The columns and bars represent means and standard errors from three independent experiments with a total of 135 pieces of callus per treatment. The double asterisk indicates significant difference in a two-tailed student's t-test (p<0.01). FIG. 1(c) shows means and standard errors for the number of GFP spots per GFP positive callus from FIG. 1(b). The double asterisk indicates significant difference in a two-tailed student's t-test (p<0.01). FIG. 1(d) shows removal of myo-inositol in culture medium enhanced perennial ryegrass transformation under conditions of cold shock and L-Gln treatments. The calluses cultured on the medium without myo-inositol showed faster recovery and more vigorous growth on selection medium containing 5.0 mg/l PPT. Typical GFP expression of calluses 2 weeks and two months after selection on medium with or without myo-inositol are also shown. Scale bar=1 mm.

FIG. 2 shows effects of callus age on transformation efficiency and InsP₆ accumulation. FIG. 2(a) shows transformation efficiency as a function of callus age. Calluses cultured on medium without myo-inositol were treated with cold shock and L-Gln in transformation as shown in FIG. 1. The calluses were subcultured once a month. The columns and bars represent means and standard errors from three independent experiments with a total of about 180 pieces of callus. The letters above the columns indicate the significant difference under a two-tailed student's t-test, p<0.05. FIG. 2(b) shows a comparison of inositol hexaphosphate (InsP₆) accumulation (μg) in callus (fresh weight, F.W.) maintained on medium containing (w/ I) or lacking myo-inositol (w/o I). The letters above the columns indicate significant difference under a two-tailed student's t-test, p<0.05. The double asterisks indicate significant difference between calluses maintained on medium with myo-inositol (w/ I) and without myo-inositol (w/o I) in a two-tailed student's t-test (p<0.01).

FIG. 3 shows effects of myo-inositol on perennial ryegrass callus. FIG. 3(a) shows characterization of four-month-old calluses cultured on medium with or without 100 mg/l myo-inositol: lignin deposit (stained with 20% phloroglucinol HCl solution) and starch accumulation (stained with 1% I₂/KI). FIG. 3(b) shows quantification of Agrobacterium tightly bound to calluses cultured on medium with 100 mg/l myo-inositol (w/ I) or without myo-inositol (w/o I) (20 mg of calluses 2 days after infection, after 3× washing, were homogenized in 1 ml liquid medium with glass beads, diluted by 10,000×, then 10 μl Agrobacterium-suspension was spread on a YEP agar plate). The experiments were performed as in 3(a). Columns and bars represent the means and standard errors of the number of bacterial colonies from 3 independent experiments. The double asterisks indicate significant difference between calluses that cultured on medium with (w/ I) or without myo-inositol (w/o I) in a two-tailed student's t-test (p<0.01).

FIG. 4 shows defense responses of perennial ryegrass callus after Agrobacterium-infection. FIG. 4(a) shows H₂O₂ generation of callus 10 days after Agrobacterium infection by staining calluses with 1 mg/l DAB (3,3′-diaminobenzidine). Four months old calluses that were subjected to three subcultures on medium with and without myo-inositol, were treated with 20 min cold-shock and 100 μM L-Gln (IGC and GC, respectively) prior to Agrobacteriu-infection, in comparison with those without the cold and L-Gln treatments (denoted w/ I and w/o I, respectively). Calluses generating H₂O₂ turned brown. The experiments were repeated at least three times to verify the observation. FIG. 4(b) shows transcripts analysis of perennial ryegrass pathogenesis related (PR) genes before and after Agrobacteriu-infection 8 days. Expression of perennial ryegrass pathogenesis related (PR) genes: LP-PR1, LP-PR2, LP-PR3, LP-PR4, LP-PR5, and LP-PR10 were analyzed by semi-quantitative RT-PCR with gene specific primers. Expression of LP-Actin1 of perennial ryegrass was used as a template loading control.

FIG. 5 shows enhanced rice callus transformation. Removal of myo-inositol enhanced rice (cv. Nipponbare) callus transformation. Rice callus generated from suspension cells, was subcultured two times (one subculture per month) on the same medium with or without 100 mg/l myo-inositol used for perennial ryegrass culture. FIG. 5(a) shows that after 3 weeks selection on medium containing 5.0 mg/l PPT, the calluses generated from the medium without myo-inositol normally had multiple GFP spots and faster growth whereas calluses generated from medium with myo-inositol had much less GFP spots and nearly no growth. Scale bar=1 mm. Combined treatment improved rice callus transformation. FIG. 5(b) shows the rice callus transformation carried out as perennial ryegrass transformation described above. The columns and bars represent means and standard errors from three independent experiments with about 60 pieces of callus in each treatment. The double asterisks indicate significant difference in a two-tailed student's t-test (p<0.01).

FIG. 6 shows characterization of rice calluses cultured on medium with (“w/ I”) or without (“w/o I”) 100 mg/l myo-inositol for two subcultures. FIG. 6(a) shows starch accumulation was revealed by staining with 1% I₂/KI. FIG. 6(b) shows lignin deposits were assayed by staining with saturated solution of phloroglucinol (Sigma) in 20% HCl solution (lower panel). Scale bar=1 mm.

FIG. 7 shows Arabidopsis respond differently to myo-inositol in Agrobacterium-mediated root fragments transformation. FIG. 7(a) shows L-Gln and cold shock treatments facilitated Arabidopsis root fragment transformation and crown gall formation, but myo-inositol had no obvious effects. The Arabidopsis seeds were germinated on B5 medium supplemented with (w/ I) or without (w/o I) MI. The L-Gln and cold shock treatments (GC) were performed the same as in the perennial ryegrass transformation. Columns and bars represent means and standard errors of gall formation from three independent experiments with about 240 root fragments in each treatment. The asterisk indicates a significant difference of the two treatments under the same myo-inostol regime in a two-tailed student's t-test (p<0.05). FIG. 7(b) shows transcripts analysis of Arabidopsis pathogenesis related (PR) genes before and 2 days after agro-infection. Expression of PR genes: AtPR1, AtPR2, AtPR3, and AtPR4 were analyzed by semi-quantitative RT-PCR with gene specific primers. Expression of AtUBQ4 was used as a template loading control.

FIG. 8 shows Arabidopsis crown galls formation, and staining test of lignin and starch in roots of Arabidopsis seedlings that were grown on medium supplied with (w/) and without (w/o) myo-inositol. FIG. 8(a) shows crown galls formation under four tested treatments. The treatments of GC and IGC gave higher percentage and bigger size of crown galls than the w/o I and w/ I treatments. Photos were taken after 30 days of agro-infection. FIG. 8(b) shows staining tests for lignin and starch accumulation in roots of one month old Arabidopsis seedling that germinated on medium supplied w/ and w/o myo-inositol. No obvious lignin and starch accumulation were detected.

### DETAILED DESCRIPTION

The present invention comprises methods of Agrobacterium based transformation of plants comprising performing callus induction and/or maintenance in a low-inositol or substantially inositol-free medium, exposing the callus explants to a cold treatment; and co-cultivating the explants and Agrobacterium in a solution comprising glutamine.

In an aspect, callus induction and/or callus maintenance is performed in a low-inositol culture medium. In an aspect, the low-inositol culture medium comprises a working concentration of not more than 100 mg/l of inositol. In a further aspect, the working concentration of inositol is not more than 75 mg/mL, not more than 50 mg/mL, not more than 40 mg/ml, not more than 30 mg/mL, not more than 25 mg/mL, not more than 20 mg/mL, not more than 15 mg/mL, not more than 10 mg/mL, not more than 5 mg/mL, not more than 4 mg/mL, not more than 3 mg/mL, not more than 2 mg/mL, or not more than 1 mg/mL. In a further aspect, callus induction and/or callus maintenance is performed in a culture medium that is substantially inositol free. In a further aspect, callus induction and/or callus maintenance is performed in a culture medium that has no inositol.

The medium for callus induction and/or maintenance may further comprise at least one plant growth regulator and/or hormone. By way of example and not limitation, the medium may comprise at least one constituent selected from the group consisting of: an N6 salt, proline, casein hydrolysate, a B-Vitamin, maltose and/or other sugars, phytagel, 2,4-D, and BAP. Other additives may be included, which will be obvious to a person of ordinary skill in the art.

In a further aspect, the callus induction medium and/or the callus maintenance medium is myo-inositol-free and further comprises N6 salts, about 1 g/l proline, about 1 g/l casein hydrolysate, about 1× Vitamin B stock, about 30 g/l maltose, about 3 g/l phytagel, about 7 mg/l 2,4-D, and about 0.05 mg/l BAP at a pH of 5.8.

In another aspect, the method comprises exposing a callus explant to a cold treatment in a second solution comprising glutamine. In an aspect, the cold treatment is performed at temperature and a time sufficient to induce “cold shock” in the explant without killing it. In another aspect, the temperature is performed at a temperature of not more than 20° C., not more than 10° C., not more than 5° C., not more than 4° C., not more than 3° C., not more than 2° C., not more than 1° C., or at about 0° C. In a further aspect, the cold treatment is performed at from 0° C.-20° C., from 0° C.-10° C., from 0° C.-5° C., from 0° C.-4° C., from 0° C.-3° C., from 0° C.-2° C., or from 0° C.-1° C. In a further aspect, at about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. In a further aspect, the cold treatment is performed for a time period of: from about 5 seconds to about 2 hours, from about 5 seconds to about 1 hour, from about 5 seconds to about 45 minutes, from about 5 seconds to about 30 minutes, from about 15 seconds to about 30 minutes, from about 30 seconds to about 30 minutes, from about 45 seconds to about 30 minutes, from about 1 minute to about 30 minutes, from about 5 minutes to about 30 minutes, from about 10 minutes to about 30 minutes, from about 15 minutes to about 30 minutes, from about 5 minutes to about 25 minutes, from about 10 minutes to about 25 minutes, from about 15 minutes to about 25 minutes, from about 5 minutes to about 20 minutes, from about 10 minutes to about 20 minutes, from about 15 minutes to about 20 minutes, and any integer from 1 minutes to 30 minutes. In a further aspect, the cold treatment is performed in an ice bath or a cold water bath. In a further aspect, the cold treatment is performed in a room or device that holds the ambient air temperature at the desired treatment temperature, such as a refrigerator or a cold room. In a further aspect, the cold treatment is performed for about 20 minutes at about 0° C.

In another aspect, the callus explant is subjected to cold treatment in the presence of a medium comprising glutamine. In an aspect, the medium comprises a concentration of glutamine of: at least 10 μM, at least 25 μM, at least 50 μM, at least 75 μM, at least 100 μM, at least 125 μM, at least 150 μM, at least 175 μM, from 10 μM to 10 mM, from 10 μM to 1 mM, from 10 μM to 750 μM, from 10 μM to 500 μM, from 10 μM to 400 μM, from 10 μM to 300 μM, from 25 μM to 750 μM, from 25 μM to 500 μM, from 25 μM to 300 μM, from 50 μM to 500 μM, from 75 μM to 500 μM, from 100 μM to 500 μM, from 50 μM to 400 μM, from 75 μM to 400 μM, from 100 μM to 400 μM, from 50 μM to 300 μM, from 75 μM to 300 μM, or from 100 μM to 300 μM. In a further aspect, the medium comprises about 100 μM glutamine. In another aspect, the medium for cold treatment may further comprise at least one plant growth regulator and/or hormone. By way of example and not limitation, the medium may further comprise at least one constituent selected from the group consisting of: an N6 salt, proline, casein hydrolysate, a B-Vitamin, maltose and/or other sugars, phytagel, 2,4-D, and BAP. Other additives may be included, which will be obvious to a person of ordinary skill in the art. In another aspect, the medium further comprises a sugar, for example, maltose. By way of example and not limitation, the sugar may be present from 5 g/L to 100 g/L. In an embodiment, the medium comprises about 100 μM glutamine and about 30 g/L of maltose.

In another aspect, Agrobaterium is co-cultivated with a callus explant in a medium comprising glutamine. In an aspect, the medium comprises a concentration of glutamine of: at least 10 μM, at least 25 μM, at least 50 μM, at least 75 μM, at least 100 μM, at least 125 μM, at least 150 μM, at least 175 μM, from 10 μM to 10 mM, from 10 μM to 1 mM, from 10 μM to 750 μM, from 10 μM to 500 μM, from 10 μM to 400 μM, from 10 μM to 300 μM, from 25 μM to 750 μM, from 25 μM to 500 μM, from 25 μM to 300 μM, from 50 μM to 500 μM, from 75 μM to 500 μM, from 100 μM to 500 μM, from 50 μM to 400 μM, from 75 μM to 400 μM, from 100 μM to 400 μM, from 50 μM to 300 μM, from 75 μM to 300 μM, or from 100 μM to 300 μM. In a further aspect, the medium comprises about 100 μM glutamine. In another aspect, the medium for cold treatment may further comprise at least one plant growth regulator and/or hormone. By way of example and not limitation, the medium may further comprise at least one constituent selected from the group consisting of: an N6 salt, proline, casein hydrolysate, a B-Vitamin, maltose and/or other sugars, phytagel, 2,4-D, and BAP. Other additives may be included, which will be obvious to a person of ordinary skill in the art.

In a further aspect, the method of Agrobaterium-based transformation of a plant comprises: (a) generating a callus explant by performing callus induction and maintenance in a plant in at least a first solution comprising not more than 100 mg/l of inositol; (b) subjecting the callus explant to a cold treatment in a second solution comprising glutamine; and (c) co-cultivating the callus explant with an Agrobacterium in a third solution comprising glutamine,

It is expected that a person of ordinary skill in the art would be able to apply the presently-disclosed methods to any species of plant capable of transformation with Agrobacterium.

In an aspect, the plant is a monocot. By way of example and not limitation, the monocot may be selected from the group consisting of a perennial ryegrass species, a rice species, and a wheat species. In another aspect, the plant is a dicot.

### EXAMPLES

1. Materials and Methods

A. Explants and Tissue Culture

Callus of perennial ryegrass and rice, and root fragments of Arabidopsis were used for transformation.

The callus of perennial ryegrass (Lolium perenne cv. Montereyll, purchased from National Seed Co., Lisle, Ill., USA) was initiated from wounded mature seeds on callus induction media NPC [N6 salts (Chu et al. (1975), Establishment of an efficient medium for anther culture of rice through comparative experiments on the nitrogen sources. Scientia Sinica 18: 659-668)+1.0 g/l proline+1.0 g/l casein hydrolysate+9.9 mg/l thiamine+9.5 mg/l pyridoxine hydrochloride+4.5 mg/l nicotinic acid+30 g/l maltose+3.0 g/l phytagel+7.0 mg/l 2,4-D+0.05 mg/l BAP, pH 5.8] with or without 100 mg/l myo-inositol. The embryo of mature seed was sliced longitudinally, and the seed was plated on callus induction medium for callus initiation. The callus was subcultured in one month interval on NPC1 medium by reducing 2,4-D to 5 mg/l and increasing BAP to 0.1 mg/l in the NPC medium.

The rice (Oryza saliva cv. Nipponbare) callus was, propagated on NPC1 medium from the suspension cells that were generated in the lab (Sivamani and Qu 2006).

Arabidopsis thaliana (Col-0 from ABRC) seeds were germinated on B5 medium (Gamborg et al. 1968) with or without myo-inositol and the root fragments (0.5 cm) were collected from one-month-old seedlings as described by Gelvin S. B. (2006), Methods in Molecular Biology, vol. 343: Agrobacterium Protocols, 2/e, volume 1 Edited by: Kan Wang© Humana Press Inc., Totowa, N.J.

All the chemicals, unless otherwise specified, were purchased from Sigma-Aldrich (St. Louis, Mo., USA).

B. Agrobacterium and Plasmid

Agrobacterium strain EHA105, harboring binary vector pTJN33-gfp, was used in perennial ryegrass and rice callus transformation. The binary plasmid pTIN33-gfp contains a bar gene driven by the CaMV 35S promoter and the Prubi3::gfp construct from plasmid pJLU11 (Lu et al. 2008) in its T-DNA region.

An overnight culture of Agrobaterium (OD₆₀₀ about 0.8) was pretreated with 200 μM acetosyringone for 2 hours in an incubator shaker at 250 rpm, 28° C. The bacterium was then collected by centrifugation at 3270 g for 15 min, and resuspended in Agrobacterium suspension medium (N6 salts+1.0 g/l proline+9.9 mg/l thiamine+9.5 mg/l pyridoxine hydrochloride+4.5 mg/l nicotinic acid+30 g/l maltose+5.0 mg/l 2,4-D+0.1 mg/l BAP+200 μM acetosyringone with or without 100 μM L-Gln, pH 5.4) to OD₆₀₀ about 0.8. The bacterium suspension was kept in an incubator shaker at 80 rpm, 28° C., for about 30 min before use.

C. Plant Transformation

For cold shock treatment, perennial ryegrass or rice calluses were immersed in 3% maltose solution on ice for 20 min prior to Agrobacterium infection. Calluses kept at room temperature were used as control.

For L-Gln treatment, various concentrations of L-Gln were added to the maltose solution and to Agrobacterium suspension. The calluses were then incubated with Agrobacterium suspension under vacuum to facilitate infection. After breaking the vacuum, the calluses were further incubated in the Agrobacterium suspension with slight agitation (80 rpm) for 20 min and then blotted dry with three layers of sterile filter paper for 30 min to remove excessive Agrobacterium suspension.

Co-cultivation was performed by placing the calluses on two layers of filter paper, soaked with 1 ml liquid NPC1 medium with addition of 200 μM acetosyringone, in petri dishes sealed with micropore surgical tape, in a 25° C. growth chamber for 4 days. The stable transformation efficiencies (the number of callus with GFP/number of callus infected×100%) and the number of GFP spots per callus were investigated a month after selection on medium containing 5 mg/l phosphinothricin.

Root fragments of one month old Arabidopsis seedlings were infected by a wild type Agrobacterium strain A208 following the procedure of Gelvin (2006). The Arabidopsis seeds were germinated on B5 medium supplemented with or without myo-inositol, to investigate the effect of myo-inositol on Arabidopsis transformation. The cold shock and L-Gln treatments were as described above. Transformation efficiency (the number of root fragments with crown gall/number of root fragments infected×100%) was counted after culturing the infected root fragments on medium with 150 mg/l timentin for a month.

D. Media and Transformation/Cultivation Schedules

Media compositions used for perennial ryegrass and rice transformation and cultivation are set forth:

TABLE 1

| Media | Composition | Time |
|---|---|---|
| Callus | N6 salts (Chu et al., 1975) + 1 g/l proline + | 1 month |
| induction | 1 g/l casein hydrolysate + 1× Vitamin | |
| | B stock** + 30 g/l maltose + phytagel 3 | |
| | g/l + 7 mg/l 2,4-D + 0.05 mg/l BAP, pH 5.8; | |
| Callus | N6 salts + 1 g/l proline + 1 g/l casein | 2 months |
| maintenance | hydrolysate + 1× Vitamin B stock + 30 g/l | |
| | maltose + phytagel 3 g/l + 5 mg/l 2,4-D + | |
| | 0.05 mg/l BAP, pH 5.8; | |
| Cold | 30 g/l maltose + glutamine 100 μM; | 20 min. |
| treatment | | |
| Agro- | N6 salts + 2 g/l proline + 1× Vitamin B | at least |
| suspension | stock + 30 at least 30 min, g/l maltose + 200 | 30 min. |
| | μM Acetosyringone + 5 mg/l 2,4-D + 0.05 | at 28°, |
| | mg/l BAP, pH 5.4; | before use |
| Co- | callus maintenance medium without | 4 days |
| cultivation | phytagel, but supplied with 200 μM | |
| | Acetosyringone, pH 5.8; | |
| Recovery/ | callus maintenance medium supplied with 5 | 1 month |
| selection | mg/l PPT, Timentin 200 mg/l, pH 5.8; | |
| Differentiation | MS (Murashige et al. (1962), A revised | 1 to 2 |
| | medium for rapid growth and bioassays with | months |
| | tobacco tissue culture. Physiol Plant 15: 473- | |
| | 497) + phytagel 3 g/l + 30 g/l maltose + 0.5 | |
| | g/l casein hydrolysate + 2 mg/l 2,4-D + 0.1 | |
| | mg/l BAP + Timentin 200 mg/l + 5 mg/l PPT, | |
| | pH 5.8; | |
| Rooting | MS + phytagel 3 g/l + 30 g/l maltose + | 2 weeks |
| | Timentin 200 mg/l + 5 mg/l PPT, pH 5.8. | |

For rice transformation, the media used for callus maintenance, Agro-suspension, cold treatment and recovery/selection were the same as for perennial ryegrass. 100 mg/l myo-inositol was added for the treatment “+1” in the callus induction and maintenance media. The 1× Vitamin B stock is as follows: Thiamine (VB1) 9.9 m/l; Pyridoxine hydrochloride (VB6) 9.5 m/l; Nicotinic acid (VB3) 4.5 m/l.

2. Characterization of the Perennial Ryegrass Callus

Calluses of perennial ryegrass (cv. Monterey II) were transformed with Agrobacterium tumefaciens strain EHA105 (pTJN33-gfp), which contains a bar selectable marker gene and a gfp reporter gene within the T-DNA region of the binary vector. Among the factors investigated to improve perennial ryegrass transformation efficiency, removal of myo-inositol from the culture medium (MI−), cold shock prior to agro-infection (C+), and addition of L-glutamine (Gln+) to the solution prior to and during the infection were found to increase transient and stable expression of the GFP reporter gene.

A. Browning of the Callus

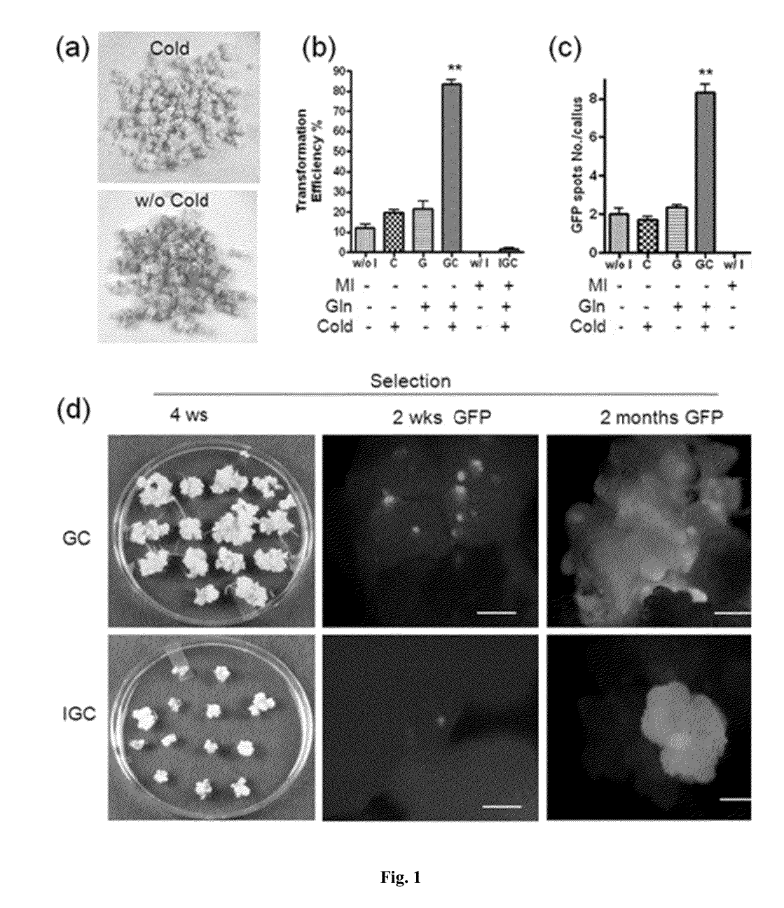

Transformed calluses were prone to turn brown after Agrobacterium infection, especially for calluses maintained on medium supplied with 100 mg/l myo-inositol. However, cold shock treatment by immersion of the callus in 3% maltose on ice for 20 min prior to infection significantly reduced the browning (FIG. 1(a)). Calluses shown in FIG. 1(a) were photographed after 4 days of co-cultivation.

B. Effect of Various Treatments on GFP Transfection Efficiency

Four-month-old calluses were cultured on medium supplied with or without 100 mg/l myo-inositol (w/ I or w/o I). The calluses were either treated with a cold shock (Cold+) or not (Cold−) for 20 min in 3% maltose solution on ice prior to infection. Addition of 100 μM L-Gln to the 3% maltose solution and bacterium suspension medium (Gln+) or not (Gln−) is another factor tested. Each condition was repeated in at least three separate experiments and the mean number of GFP spots per GFP positive callus was calculated. Results are shown in FIGS. 1(b) and 1(c) and Table 2.

As shown in FIG. 1(b), although each of the three factors had a positive effect on transformation, the combination of the three (labeled as GC) improved the transformation efficiency of perennial ryegrass. As investigated a month after selection, compared to the callus from myo-inositol containing medium (w/ I), the treatment without myo-inositol (w/o I) increased transformation efficiency from 0 to 11.8% based on the number of callus with stably transformed GFP spots. Transformation efficiency was improved to about 20% when cold shock (C) or L-Gln (G) treatment was added. Combination of the three treatments (GC) resulted in a much higher transformation efficiency of 83.7%.

A similar trend was observed when GFP spots per GFP positive callus were counted: GC treatment had the highest number of GFP callus spots, about 8 per piece of callus. The other treatments (w/o I, C, G, IGC) only had one or two GFP spots per callus. Results are shown in FIG. 1(c).

During callus culture stage and right after infection, callus growth and morphology looked similar when the callus was cultured on medium with or without myo-inositol. However, under selection of 5.0 mg/l phosphinothricin (PPT) after agro-infection, calluses from GC treatment had faster recovery and more vigorous growth. Significant difference between GC and IGC in the size of callus was observed 4 weeks after selection. Multiple GFP spots were observed two weeks after selection in the GC treated callus without myo-inositol. In contrast, the callus maintained on the medium with myo-inositol showed sluggish growth and cell death (IGC=MI+, Gln+, cold+) and had many fewer GFP spots. Two months after selection, the GFP positive calluses with GC treatment showed robust growth and were ready for differentiation, but not the ones from IGC treatment. Results are shown in FIG. 1(d).

Data from several experiments are compiled in Table 2 below. Transformation results were recorded about 35 days post-transfection. “I” indicates callus grown in media either lacking (−) or including (+) 100 mg/L myo-inositol. “C” indicates that the callus was either nontreated (−) or exposed to a 20 min cold treatment prior to infection (+). “G” indicates that media either lacked (−) or including (+) 100 μM L-glutamine. A callus was determined to be GFP+ if it contained at least one GFP spot. Transformation efficiency (%) was calculated as the number of callus containing at least GFP spot divided by the initiated Callus No. multiplied by 100. GFP spots per calli was calculated as the total GFP spots divided by the number of GFP+ calli.

TABLE 2

| | | | Number of | Number of | | |
|---|---|---|---|---|---|---|
| Treatment | | | Calli | GFP⁺ | GFP Spots | Transformation |
| I | C | G | tested | Calli | per Calli | efficiency (%) |
| − | + | + | 45 | 39 | 8.9 | 86.7 |
| | | | 45 | 38 | 8.6 | 84.4 |
| | | | 30 | 24 | 7.5 | 80.0 |
| | | | 30 | 25 | 7.4 | 83.3 |
| | | | 35 | 28 | 6.5 | 80.0 |
| − | − | + | 45 | 9 | 1.6 | 20.0 |
| | | | 30 | 5 | 1.6 | 16.7 |
| | | | 45 | 10 | 2 | 22.2 |
| − | + | − | 30 | 4 | 2.5 | 13.3 |
| | | | 45 | 11 | 2.5 | 24.4 |
| | | | 45 | 12 | 2 | 26.7 |
| − | − | − | 45 | 4 | 2.5 | 8.9 |
| | | | 45 | 5 | 1.4 | 11.1 |
| | | | 45 | 7 | 2.1 | 15.6 |
| + | + | + | 45 | 1 | 1 | 2.2 |
| | | | 45 | 1 | 1 | 2.2 |
| | | | 45 | 0 | 0 | 0.0 |
| + | − | − | 45 | 0 | 0 | 0.0 |
| | | | 45 | 0 | 0 | 0.0 |
| | | | 45 | 0 | 0 | 0.0 |

C. Effect of Age on Transformation Efficiency

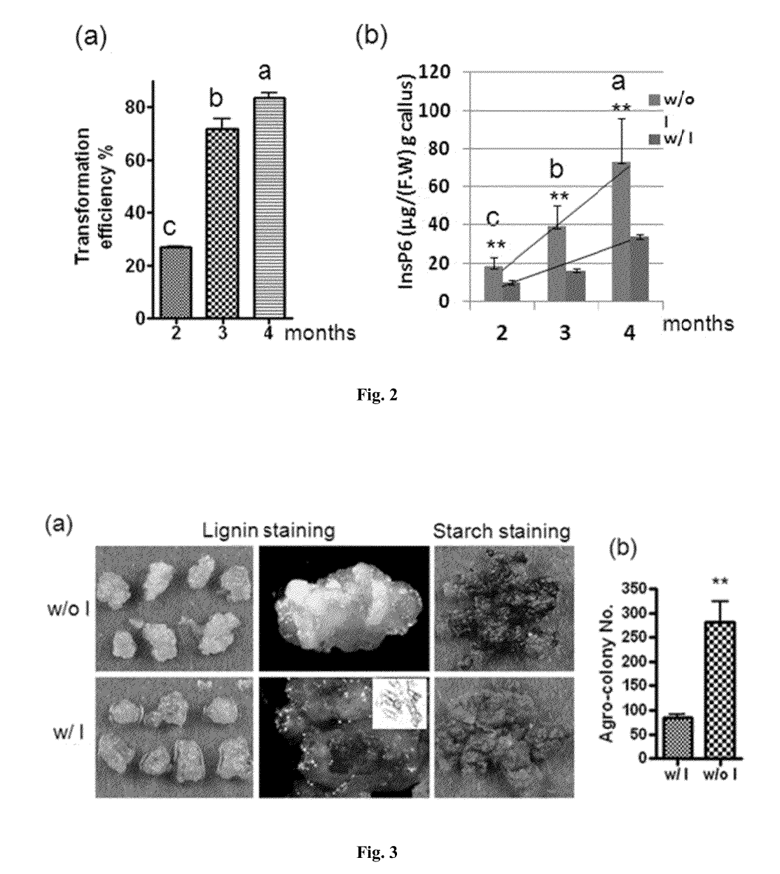

Calluses with three subcultures (about 4-month-old) were used for transformation without myo-inositol and with cold shock and L-glutamine as in Example 2B. The calluses were subcultured once a month and transformation efficiency calculated at each subculture. Results are shown in FIG. 2(a). The columns and bars represent means and standard errors from three independent experiments with a total of about 180 pieces of callus. The letters above the columns indicate the significant difference under a two-tailed student's t-test, p<0.05.

As shown at FIG. 2(a), callus of 2, 3, and 4 months old (corresponding to one, two and three subcultures), had transformation efficiency of 27%, 71.8%, and 83.7%, respectively.

D. Effect of Myo-Inositol on Accumulation of InsP₆

Calluses with three subcultures (about 4-month-old) were used for transformation with cold shock and L-glutamine as in Example 2B and either with or without myo-inositol. The calluses were subcultured once a month. Inositol hexaphosphate (InsP6) accumulation was compared in transformed callus of 2, 3, and 4 months old. Accumulation of inositol hexaphosphate (InsP₆) was analyzed with high-performance liquid chromatography (HPLC) as described by Philliphy et al. (2003), Ion chromatography of phytate in roots and tubers. J Agric Food Chem 51:350-353. An InsP₆ standard sample (Sigma, St. Louis, Mo. (P8801) as a reference. Briefly, 2 g of callus was ground to fine powder with mortar and pestle in liquid nitrogen, transferred to a 15 ml tube, and was added with 3 ml 0.75 M HCl. The mixture was boiled for 15 min and centrifuged at 3700 g for 5 min. Two ml supernatant was centrifuged again at 16,000 g for 10 min. The supernatant was passed through a C18 SEP-PAK® column (Waters Co., Milford, Mass., USA) that was pre-eluted with 2 ml methanol and 5 ml H₂O. The elution was collected, filtered through a 0.45 μm filter, and analyzed by isocratic ion chromatography.

Results are shown at FIG. 2(b). Data are represented as μg of InsP6 per fresh weight of callus in grams (F.W.). The letters above the columns indicate a significant difference under a two-tailed student's t-test, p<0.05. Double asterisks indicate significant difference between calluses maintained on medium with myo-inositol (w/ I) and without myo-inositol (w/o I) in a two-tailed student's t-test (p<0.01).

As shown at FIG. 2(b), phytate (inositol hexaphosphate, or InsP6) accumulation in the cultured callus increased significantly with age of the callus, while removal of myo-inositol from the culture medium more than doubled the InsP6 levels in callus of all ages tested, indicating that both callus age and supplement of myo-inositol to the medium affected inositol phosphate metabolism. Considering the key role of myo-inositol in the biosynthesis of various inositol phosphates including InsP6, and the various functions of inositol phosphates in signal transduction, chromatin remodeling, RNA exporting and DNA repairing, it is possible that the altered metabolism of inositol phosphates could affect genetic transformation frequency.

E. Effect of Myo-Inositol on Lignin Deposit and Starch Accumulation

Four-month-old callus (which were used for transformation) growing on medium supplemented with or without myo-inositol were tested for lignin deposit and starch accumulation. Lignin in callus cell walls was assayed by staining with 20% phloroglucinol HCl solution as described by Stange et al. (2002), Studies on the phloroglucinol-HCl reactive material produced by squash fruit elicited with pectinase: isolation using hydrolytic enzymes and release of p-coumaryl aldehyde by water reflux. Physiological and Molecular Plant Pathology 60, pp. 283-291. Starch accumulation is tested by staining with 1% I₂/KI. Results are shown at FIG. 3(a).

As shown in FIG. 3(a), callus growing on medium supplemented with myo-inositol had lignin deposit in cell walls and no obvious starch accumulation. In contrast, callus grown on medium without supplement of myo-inositol did not show appreciable lignin accumulation and had substantial amount of starch (FIG. 3(a)). This suggests that supplement of the culture medium with myo-inositol promotes development of secondary cell wall and affects carbohydrate metabolism in the cultured callus.

F. Effect of Myo-Inositol on Tight Binding of Agrobacterium to Calli

The tight binding of Agrobacterium was evaluated in calluses cultured on medium with 100 mg/l myo-inositol (w/ I) or without myo-inositol (w/o I) 2 days after infection. Twenty mg of infected callus were mixed with 1.0 ml Agrobacterium suspension liquid medium. After vortexing for 1 min, the liquid was removed with a pipette to get rid of the Agrobacterium in loose-binding. This operation was repeated 3 times. Then, 20 μl glass beads (450-600 μm in diameter) was added to the callus in 1 ml Agrobacterium suspension liquid medium and vortexed for 3 min to release the bacteria tightly bound to the callus cell walls. The bacterium suspension was diluted 10,000 times, and 10 μl of the final dilution was mixed with 50 μl sterilized water and spread on YEP solid medium containing proper antibiotics. Two days later, the colony numbers were counted. Each colony represents a single bacterium tightly bound to the callus cell wall.

Results are shown at FIG. 3(b). Columns and bars represent the means and standard errors of the number of bacterial colonies from 3 independent experiments. The double asterisks indicate significant difference between calluses that cultured on medium with (w/ I) or without myo-inositol (w/o I) in a two-tailed student's t-test (p<0.01). As can be seen, about four times more Agrobacterium were observed to tightly bind to callus when myo-inositol was removed from the callus maintaining medium, indicating that the metabolic changes caused by the removal of myo-inositol may facilitate Agrobacterium binding to plant cells and its infection.

G. Effect of Myo-Inositol on Defense Responses of Perennial Ryegrass Callus After Agrobacterium-Infection

(1) Histochemistry Test of H₂O₂

Four months old calluses that were subjected to three subcultures on medium with and without myo-inositol, were treated with 20 min cold-shock and 100 μM L-Gln (IGC and GC, respectively) prior to Agrobacterium-infection, in comparison with those without the cold and L-Gln treatments (denoted w/ I and w/o I, respectively). Generation of H₂O₂ was analyzed by DAB (3,3′-diaminobenzidine) staining, as described by Thordal-Christensen et al. (1997), Subcellular localization of H2O2 in plants. H2O2 accumulation in papillae and hypersensitive response during the barley-powdery mildew interaction. Plant J. 11: 1187-1194. The callus tissue was sampled 10 days after infection, and placed in 1 mg/ml DAB solution for 15 h. To develop the reddish-brown coloration of DAB polymers, the stained materials were placed in boiling 95% Ethanol for 10 min. Calluses generating H₂O₂ turned brown. The experiments were repeated at least three times to verify the observation.

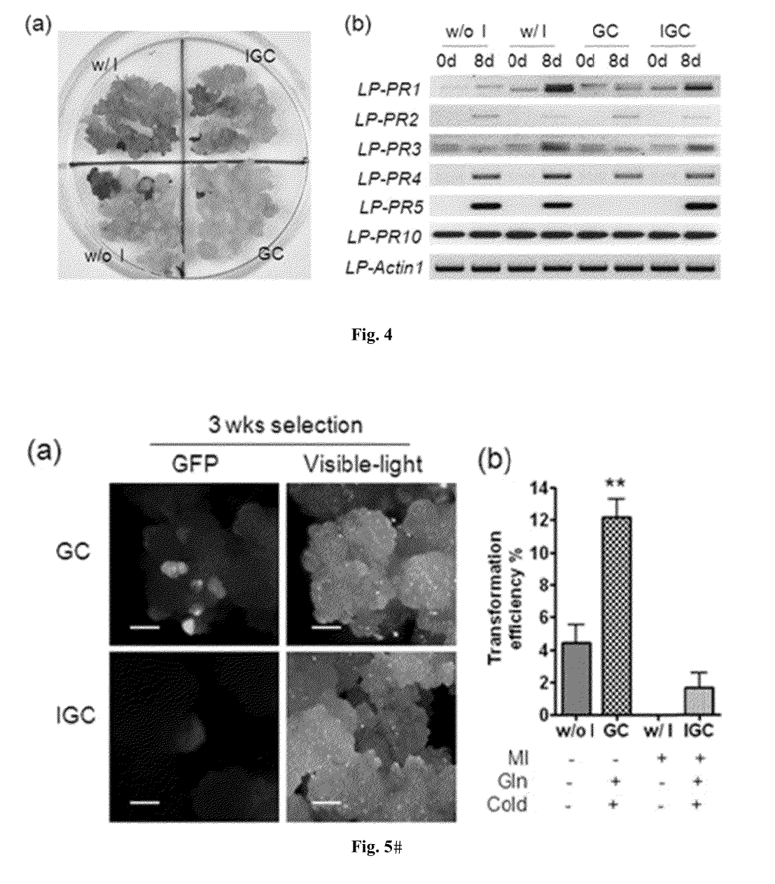

Results are shown at FIG. 4(a). Most of the calluses grown on myo-inositol-containing medium without cold and L-glutamine treatments (w/ I) turned brown. For such calluses with cold and Gln treatments (IGC), or calluses growing on inositol-free medium without cold and Gln treatments (w/o I), a majority of the calluses did not turn brown, indicating that fewer cells generated H₂O₂. For the calluses with the combined treatments (GC), little H₂O₂ was detected. The results further revealed the treatments mitigate the plant defense system triggered by Agrobacterium-infection.

(2) Expression of Plant Defense-Related Genes

Expression of perennial ryegrass pathogenesis related (PR) genes, LP-PR1, 2, 3, 4, 5 and 10, was analyzed by semi-quantitative RT-PCR before, and 8 days after, Agrobacterium infection. Total RNA of perennial ryegrass callus, before and 8 days after agro-infection, was isolated using TRIZOL® reagent as described by Invitrogen (Carlsbad, Calif., USA). RT-PCR was performed with the SUPERSCRIPT III® One-Step RT-PCR system (Invitrogen). Gene specific primers were used in a 10 μl reaction, with the annealing temperature of 53, 55 or 60° C. for 20 sec in reaction based on Tm report. 100 ng total RNA, and 25 reaction cycles were used in the reactions. The Actin1 gene of perennial ryegrass was used as an internal control for equal RNA sample loading. Each RT-PCR was performed three times. Two independent experiments were performed. The amplified DNA products were separated on 1.2% (w/v) agarose gels. Each gene was tested at least three times in different transformation experiments to verify expression pattern. Primer sequences are shown at Table 3.

TABLE 3

| SEQ | | |
|---|---|---|
| ID | Primer name and | |
| NO | sequence | Gene |
| 1 | LpPR1_f: | LpPR1 |
| | ACGGCGAGAACATCTTCTGG | |
| 2 | LpPR1_r: | |
| | CGCCGAGGTTGTTGTCGCAG | |
| 3 | LpPR2_f: | LpPR2 |
| | AGGTGAACGGCAGCGATCC | |
| 4 | LpPR2_r: | |
| | ACCCGTCTTGGCAAGGTACAG | |
| 5 | LpPR3_f: | LpPR3 |
| | ACCCCGACTCTTCCCTT | |
| 6 | LpPR3_r: | |
| | ATGATGTTGGTGATCACGCCG | |
| 7 | LpPR4_f: | LpPR4 |
| | CCCGCGGCCAGGAATCG | |
| 8 | LpPR4_r: | |
| | GGCCCTGCTGCACTCCCA | |
| 9 | LpPR5_f: | LpPR5 |
| | GCCAGTGGGCGGTGGTAGGC | |
| 10 | LpPR5_r: | |
| | ATGCCGATGTTGAACCCGTC | |
| 11 | LpPR10_f: | LpPR10 |
| | GACTGGCACAATCTGGCACCC | |
| 12 | LpPR10_r: | |
| | CTTGGCCACACTCCCACCG | |
| 13 | LpActin1_f: | LpActin1 |
| | GCTGTTTTCCCTAGCATTGTTGG | |
| 14 | LpActin1_r: | |
| | ATAAGAGAATCCGTGAGATCCCG | |

Exemplary results are shown at FIG. 4(b). Expression of LP-PR1, LP-PR2, LP-PR3, LP-PR4 and LP-PR5 was induced after Agrobacterium-infection, whereas LP-PR10 expression was constant and unaffected by the infection. Among the six PR genes investigated, both LP-PR1 and LP-PR3 seem to have two isoforms and their expression was clearly affected by myo-inositol in the medium. The induction of both genes by Agrobaceterium-infection was much reduced in the treatments of lacking myo-inositol (w/o I and GC) when compared to the treatments containing myo-inositol in medium (w/ I and IGC), and the expression pattern of the two isoforms was also altered. For LP-PR5, its expression pattern was not affected much by removal of myo-inositol only (w/o I) nor by the treatments of w/ I and IGC, but LP-PR5 induction was mostly suppressed by the combined treatment (GC) without myo-inositol, suggesting a synergistic effect of the combined treatment on LP-PR5 induction. These results indicate that all the three treatments do affect plant defense reactions to Agrobacterium infection.

3. Characterization of Rice Callus Transformation

To test whether the combined treatments also promote transformation of other monocot species, the rice cell line of cv. Nipponbare (Sivamani and Qu 2006) was cultured on the same medium used for perennial ryegrass callus maintenance as described at Example 2B. No obvious morphological difference was observed between the calluses growing on the medium supplied with or without myo-inositol in the four tested treatments as did on perennial ryegrass (GC, IGC, w/o I and w/ I). After 4 days of co-cultivation, calluses of similar size were transferred to selection medium containing 5 mg/l PPT. Three weeks after selection, the calluses were inspected for GFP transfection efficiency. As shown at FIG. 5(a), plants grown on the medium without myo-inositol had multiple GFP spots and showed vigorous growth, whereas the calluses generated from the medium with myo-inositol had many fewer GFP spots and only sluggish growth.

Stable transformation of callus was counted one month after selection based on GFP expression. Results are shown at FIG. 5(b). Statistical analysis showed that the GC treatment resulted in an average transformation efficiency of 12.2% from three independent experiments, highly significantly different from other treatments (p<0.01, FIG. 5(b)). The w/o I treatment had a transformation efficiency of 4.4%. The IGC treatment had a transformation efficiency of 1.7% whereas no callus from inositol-containing medium (w/ I) without GC treatment was transformed.

Additional transformation results were recorded about 35 days post-transfection. “I” indicates callus grown in media either lacking (−) or including (+) 100 mg/L myo-inositol. “C” indicates that the callus was either nontreated (−) or exposed to a 20 min cold treatment prior to infection (+). “G” indicates that media either lacked (−) or including (+) 100 μM L-glutamine. A callus was determined to be GFP+ if it contained at least one GFP spot. Transformation efficiency (%) was calculated as the number of callus containing at least GFP spot divided by the initiated Callus No. multiplied by 100. GFP spots per calli was calculated as the total GFP spots divided by the number of GFP+ calli. Data are compiled in Table 4 below.

TABLE 4

| | | | Number of | Number of | | |
|---|---|---|---|---|---|---|
| Treatment | | | Calli | GFP⁺ | GFP Spots | Transformation |
| I | C | G | tested | Calli | per Calli | efficiency (%) |
| − | + | + | 60 | 8 | 3.0 | 13.3 |
| | | | 60 | 8 | 2.8 | 13.3 |
| | | | 60 | 6 | 1.8 | 10.0 |
| + | + | + | 60 | 1 | 1.0 | 1.7 |
| | | | 60 | 2 | 2.5 | 3.3 |
| | | | 60 | 0 | 0.0 | 0.0 |
| − | − | − | 30 | 2 | 1.5 | 6.7 |
| | | | 30 | 1 | 2.0 | 3.3 |
| | | | 60 | 2 | 3.5 | 3.3 |
| + | − | − | 30 | 0 | 0.0 | 0.0 |
| | | | 30 | 0 | 0.0 | 0.0 |
| | | | 60 | 0 | 0.0 | 0.0 |

The results suggest the transformation response of rice is similar to that of perennial ryegrass: removal of myo-inositol from the medium improves transformation efficiency, and cold shock and L-Gln treatments further increase transformation efficiency.

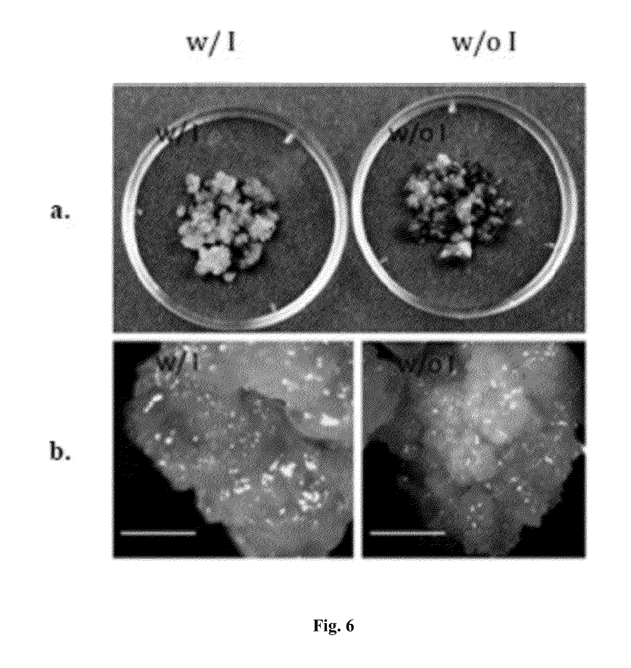

In addition, three months old rice calluses that were cultured on medium supplied with or without myo-inositol were assayed for lignin and starch accumulation as in Example 2E. Results are shown at FIGS. 6(a) and 6(b). As shown at FIGS. 6(a) and 6(b), three months old rice calluses that were cultured on medium supplied with or without myo-inositol for two subcultures showed similar patterns in lignin and starch accumulation as perennial ryegrass calluses.

4. Characterization of Arabidopsis Transformation

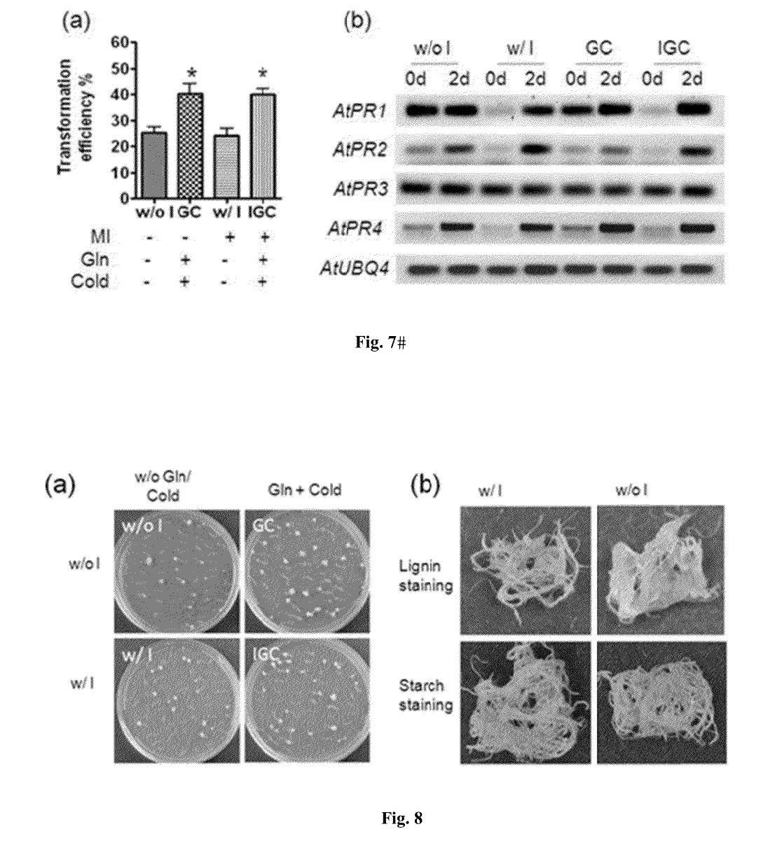

A dicot plant, Arabidopsis thaliana, was also evaluated using a wild-type Agrobacterium strain, A208 (FIG. 8(a)). Arabidopsis seeds were germinated on B5 medium supplemented with (w/ I) or without (w/o I) myo-inositol. Root fragments of one-month-old seedlings that germinated on medium with or without myo-inositol (w/ I or w/o I) were infected for crown gall formation. L-glutamine and cold shock treatments (GC) were performed the same as in the perennial ryegrass transformation in Example 2A and transfection efficiency was calculated, and crown gall formation was photographed after 30 days of agro-infection.

Results are shown in FIGS. 7(a) and 8(a). Columns and bars in 7(a) represent means and standard errors of gall formation from three independent experiments with about 240 root fragments in each treatment. The asterisk in FIG. 7a indicates a significant difference of the two treatments under the same myo-inostol regime in a two-tailed student's t-test (p<0.05). As can be seen, the combination of cold shock and 100 μM L-glutamine promoted crown gall formation (GC and IGC) significantly. Compared to the treatments of w/o I and w/ I, the treatments of GC and IGC improved transformation by about 60%. While the treatments of L-Gln and cold increased transformation efficiency significantly, adding Myo-inositol alone or in addition to the GC treatment had no effect on Arabidopsis root fragment transformation or crown gall formation, indicating that presence or absence of myo-inositol in the medium had no obvious effect on Arabidopsis root fragment transformation and crown gall formation. Additionally, as can be seen at FIG. 8(a), the treatments including cold shock and L-glutamine gave higher percentage and bigger size of crown galls than the treatments lacking cold shock and L-glutamine.

Additionally, lignin deposit and starch accumulation were measured. As shown at FIG. 8(b), no differences in lignin and starch accumulation could be observed in the roots of Arabidopsis seedlings grown with or without myo-inositol.

Expression of Arabidopsis pathogenesis related (PR) genes: AtPR1, 2, 3 and 4 was also evaluated before and 2 days after agro-infection using semi-quantitative RT-PCR and are shown in FIG. 7(b). Total RNA was isolated before and 8 days after agro-infection using TRIZOL® reagent as described by Invitrogen (Carlsbad, Calif., USA). RT-PCR was performed with the SUPERSCRIPT III® One-Step RT-PCR system (Invitrogen). Gene specific primers were used in a 10 μl reaction, with the annealing temperature of 53, 55 or 60° C. for 20 sec in reaction based on Tm report. 100 ng total RNA, and 25 reaction cycles were used in the reactions. The AtUBQ4 gene was used as an internal control for equal RNA sample loading. Each RT-PCR was performed three times. The amplified DNA products were separated on 1.2% (w/v) agarose gels. Primer sequences are shown at Table 5.

TABLE 5

| SEQ | | |
|---|---|---|
| ID | Primer name and | |
| NO | sequence | Gene |
| 15 | AtUBQ4_f: | AtUBQ4 |
| | GCTTGGAGTCCTGCTTGGACG | |
| 16 | AtUBQ4_r: | |
| | CGCAGTTAAGAGGACTGTCCGGC | |
| 17 | AtPR1_f: | AtPR1 |
| | AAGGGTTCACAACCAGGCAC | |
| 18 | AtPR1_r: | |
| | CACTGCATGGGACCTACGC | |
| 19 | AtPR2_f: | AtPR2 |
| | GGGACGGCTCTCGTGGCTACC | |
| 20 | AtPR2_r: | |
| | CGCGCGTTATCGAAACTCGCGG | |
| 21 | AtPR3_f: | AtPR3 |
| | GACGCCGACCGTGCCGCCGGG | |
| 22 | AtPR3_r: | |
| | CGGCGACTCTCCCGTCTTGGCC | |
| 23 | AtPR4_f: | AtPR4 |
| | GGACCAATGCAGCAACGGAGGC | |
| 24 | AtPR4_r: | |
| | GGCTGCCCAATGAGCTCATTGCC | |

Exemplary results are shown at FIG. 8(b). In the absence of myo-inositol (w/o I and GC), AtPR1 expression was already high before infection and was not further induced by the infection, whereas its expression was low in the presence of myo-inositol (w/ I and IGC) before infection, and was induced by the infection. Two days after Agrobacterium-infection, expression of AtPR2 and AtPR4 was induced in all the tested treatments except that the induction of AtPR2 in GC treatment appeared to be weaker when compared to IGC. In contrast, expression of AtPR3 was constant and unaffected by either supplementary myo-inostol or Agrobacterium-infection.

What is claimed is:

1. A method of Agrobaterium-based transformation of a plant comprising:
generating a callus explant by performing callus induction and maintenance in a plant in at least a first solution comprising less than about 100 mg/l of inositol;
subjecting the callus explant to a cold treatment in a second solution comprising glutamine; and
co-cultivating the callus explant with an Agrobacterium in a third solution comprising glutamine.

2. The method of claim 1, wherein callus induction is performed in a callus induction medium and callus maintenance is performed in a callus maintenance medium, and wherein the callus induction medium and callus maintenance medium comprise less than about 100 mg/l of inositol.

3. The method of claim 2, wherein the callus induction medium and/or the callus maintenance medium comprises at least one constituent selected from the group consisting of: an N6 salt or any plant culture media, proline, casein hydrolysate, a B-Vitamin, maltose, phytagel, 2,4-D, BAP, and other plant growth regulators/hormones.

4. The method of claim 3, wherein the callus induction medium and/or the callus maintenance medium further comprises N6 salts, about 1 g/l proline, about 1 g/l casein hydrolysate, about 1× Vitamin B stock, about 30 g/l maltose, about 3 g/l phytagel, about 7 mg/l 2,4-D, and about 0.05 mg/l BAP at a pH of 5.8.

5. The method of claim 2, wherein the callus induction medium and/or the callus maintenance medium is essentially inositol-free.

6. The method of claim 1, wherein the second solution comprises from 10 μM to about 10 mM of glutamine.

7. The method of claim 6, wherein the second solution comprises about 100 μM to about 300 μM of glutamine.

8. The method of claim 1, wherein the second solution further comprises 5 g/l to 100 g/l of maltose or other sugars.

9. The method of claim 8, wherein the second solution comprises about 30 g/l of maltose.

10. The method of claim 1, wherein the cold treatment is performed before an agroinfection and is performed for about 5 sec to about 2 hr.

11. The method of claim 10, wherein the cold treatment is performed for about 5 minutes to about 45 minutes.

12. The method of claim 8, wherein the cold treatment is performed for about 20 minutes.

13. The method of claim 1, wherein the second solution comprises about 100 μM glutamine, and wherein in the cold treatment the callus explant is treated on ice for about 20 minutes.

14. The method of claim 1, wherein the third solution comprises from about 10 μM to about 10 mM of glutamine.

15. The method of claim 14, wherein the third solution comprises about 100 μM of glutamine.

16. The method of claim 1, wherein the plant is a monocot or a dicot.

17. The method of claim 1, wherein the plant is a perennial ryegrass, a rice, or a wheat.

18. A callus induction medium and/or callus maintenance medium, comprising:
less than about 100 mg/l of inositol, and
optionally at least one constituent selected from the group consisting of: an N6 salt or any plant culture media, proline, casein hydrolysate, a B-Vitamin, maltose, phytagel, 2,4-D, BAP, and other plant growth regulators/hormones.

19. The callus induction medium and/or the callus maintenance medium of claim 18, further comprising N6 salts, about 1 g/l proline, about 1 g/l casein hydrolysate, about 1× Vitamin B stock, about 30 g/l maltose, about 3 g/l phytagel, about 7 mg/l 2,4-D, and about 0.05 mg/l BAP at a pH of 5.8.

20. The callus induction medium and/or the callus maintenance medium of claim 18, wherein the callus induction medium and/or callus maintenance medium is essentially inositol-free.
